(19) United States
(12) Patent Application Publication
(10) Pub. No.: US 2013/0196927 A1
(43) Pub. Date: Aug. 1, 2013

(54) Smac Mimetic Therapy

(71) Applicants: Christopher BENETATOS, Downingtown, PA (US); Srinivas CHUNDURU, West Chester, PA (US); Stephen CONDON, Glenmoore, PA (US); Mark McKINLAY, Phoenixville, PA (US); Yasuhiro MITSUUCHI, Melrose Park, PA (US)

(72) Inventors: Christopher BENETATOS, Downingtown, PA (US); Srinivas CHUNDURU, West Chester, PA (US); Stephen CONDON, Glenmoore, PA (US); Mark McKINLAY, Phoenixville, PA (US); Yasuhiro MITSUUCHI, Melrose Park, PA (US)

(21) Appl. No.: 13/751,959

(22) Filed: Jan. 28, 2013

Related U.S. Application Data
(60) Provisional application No. 61/591,459, filed on Jan. 27, 2012.

(51) Int. Cl. C07K 5/02

(52) U.S. Cl. CPC C07K 5/02
USPC 514/18.9; 530/323; 514/19.3; 514/19.4; 514/19.6; 514/19.8; 435/7.1; 514/19.5

(57) ABSTRACT

A Smac mimetic therapy wherein the Smac mimetic is selected and developed based at least in part on its cIAP degradation properties.

### CROSS-REFERENCE TO RELATED APPLICATION

This application claims priority to and the benefit of U.S. Provisional Application No. 61/591,459, entitled, “Smac Mimetic Therapy” filed Jan. 27, 2012, the entire contents of which are hereby incorporated by reference in its entirety.

### FIELD OF THE INVENTION

This invention is in the field of compositions and methods to treat proliferative disorders including cancers.

### BACKGROUND OF THE INVENTION

Inhibitors of Apoptosis Proteins (IAPB) are naturally occurring intra-cellular proteins that suppress caspase-dependent apoptosis. SMAC, also known as DIABLO, is another intracellular protein that functions to antagonize, i.e., inhibit the activity of, IAPB. In normal healthy cells, SMAC and IAPB function together to maintain the viability of healthy cells. However, in certain disease states, e.g., cancers and other proliferative disorders, IAPB are not adequately antagonized and therefore prevent apoptosis and cause or exacerbate abnormal proliferation and survival.

Smac mimetics, also known as IAP antagonists, are synthetic small molecules that mimic the structure and IAP antagonist activity of the four N-terminal amino acids of SMAC. (Smac mimetics are sometimes referred to as IAP antagonists.) When administered to animals suffering proliferative disorders, the Smac mimetics antagonize IAPB, causing an increase in apoptosis among abnormally proliferating cells. Various Smac mimetics are in development for use in the treatment of proliferative disorders.

Examples of SMAC peptidomimetics are those disclosed in, without limitation, U.S. Pat. No. 7,244,851, U.S. Pat. No. 7,345,081, U.S. Pat. No. 7,419,975, U.S. Pat. No. 7,456,209, U.S. Pat. No. 7,517,906, U.S. Pat. No. 7,547,724, U.S. Pat. No. 7,579,320, U.S. Pat. No. 7,589,118, U.S. Pat. No. 7,674,787, U.S. Pat. No. 7,772,177, U.S. Pat. No. 7,932,382, U.S. Pat. No. 7,795,298, U.S. Pat. No. 7,960,372, U.S. Pat. No. 7,985,735, U.S. Pat. No. 7,989,441, U.S. Pat. No. 8,063,095, U.S. Pat. No. 8,143,426, U.S. Pat. No. 8,163,792, U.S. Pat. No. 8,207,183, U.S. Pat. No. 8,247,557, U.S. Pat. No. 8,283,372, US20120094917, WO2010138666, WO2012052758, etc.

TNF receptor-associated factor 2 is a member of the TNF receptor associated factor (TRAF) protein family. TRAF proteins associate with, and mediate the signal transduction from members of the TNF receptor superfamily. This protein directly interacts with TNF receptors, and forms complexes with other TRAF proteins. The protein complex formed by TRAF2 alone or by TRAF2 and TRAF1 interacts with cIAP-1 and cIAP-2, and functions as a mediator of the anti-apoptotic signals from TNF receptors. This complex triggers the activation of NF-kB pathway.

### SUMMARY OF THE INVENTION

This invention, in one aspect, relates to a method of treating a proliferative disorder, such as a cancer or a benign proliferative disorder or an autoimmune disorder mediated or exacerbated by defective regulation of apoptosis, in a mammalian subject, e.g., a human patient, by selecting and then internally administering to the subject an effective amount of a Smac mimetic, i.e., a small molecule that binds to a BIR domain of at least one IAP, e.g., XIAP, cIAP-1, and cIAP-2, leading to ubiquitination (also, referred to as ubiquitylation) and degradation of the cIAPs, wherein said Smac mimetic:
degrades cIAP-1 not bound to TRAF2 (non TRAF2-bound, e.g., “cytoplasmic” cIAP-1 or “free” cIAP-1)
degrades cIAP-1 bound to TRAF2,
degrades cIAP-2 bound to TRAF2;
does not degrade cIAP-2 not bound to TRAF2 (non TRAF2-bound cIAP-2, e.g., “cytoplasmic” cIAP-2 or “free” cIAP-2) or degrades cIAP-2 not bound to TRAF2 to a lesser extent than cIAP-2 bound to TRAF2.

Thus, in one aspect, such method of treating a proliferative disorder in a mammalian subject, e.g., a human patient, comprises:

a) assaying a Smac mimetic in a cIAP degradation assay to determine whether or not the Smac mimetic induces degradation of cIAP-2 not bound to TRAF2 and of cIAP-2 bound to TRAF2
and, if the Smac mimetic degrades cIAP-2 bound to TRAF2 and either does not degrade cIAP-2 not bound to TRAF2 or degrades cIAP-2 not bound to TRAF2 to a lesser extent than cIAP-2 bound to TRAF2, then
b) internally administering an effective amount of a Smac mimetic having the same chemical structure as the assayed Smac mimetic to the patient.

In another illustrative embodiment, the invention is such method that also comprises assaying the Smac mimetic to determine the ability of the Smac mimetic to induce degradation of cIAP-1 not bound to TRAF2 and cIAP-1 bound to TRAF2 and, if the Smac mimetic degrades cIAP-1 not bound to TRAF2 and cIAP-1 bound to TRAF2, but either does not degrade cIAP-2 not bound to TRAF2 or degrades cIAP-2 not bound to TRAF2 to a lesser extent than cIAP-2 bound to TRAF2, then internally administering a Smac mimetic to the subject.

In another illustrative embodiment of the invention, the invention comprises, for example a method of preparing a pharmaceutical composition comprising a Smac mimetic for the treatment of a patient suffering a proliferative disorder, said method comprising assaying and selecting a Smac mimetic on the basis of its ability to degrade cIAP-1 not bound to TRAF2, cIAP-1 bound to TRAF2 and cIAP-2 bound to TRAF2, while demonstrating less degradation of cIAP-2 not bound to TRAF2 relative to TRAF2 bound cIAP-2 and formulating a Smac mimetic having the same structure as the Smac mimetic demonstrating such degradation properties into a pharmaceutical composition;

In another illustrative embodiment of the invention, the invention comprises, for example a method of obtaining regulatory approval for a Smac mimetic for use in the treatment of a proliferative disorder, said method comprising assaying a Smac mimetic for the ability to degrade cIAP-1 not bound to TRAF2, cIAP-1 bound to TRAF2 and cIAP-2 bound to TRAF2, while demonstrating less degradation of cIAP-2 not bound to TRAF2 relative to TRAF2-bound cIAP-2 and, if the Smac mimetic has such characteristic, then submitting such assay results to the regulatory agency as part of a dossier that forms the basis for a request for regulatory approval of such Smac mimetic;

In another illustrative embodiment of the invention, the invention comprises, for example a method of screening for a Smac mimetic for use in the treatment of a proliferative disorder in a patient, said method comprising assaying a Smac mimetic to determine the ability of the Smac mimetic to induce degradation of cIAP-1 not bound to TRAF2, cIAP-1 bound to TRAF2 and cIAP-2 bound to TRAF2, while demonstrating less degradation of cIAP-2 not bound to TRAF2 relative to TRAF2-bound cIAP-2.

In another illustrative embodiment of the invention, the invention comprises, for example a method of marketing a Smac mimetic for use in the treatment of a proliferative disorder, said method comprising assaying a Smac mimetic to determine the ability of the Smac mimetic to induce degradation of cIAP-1 not bound to TRAF2, cIAP-1 bound to TRAF2 and cIAP-2 bound to TRAF2, while demonstrating less degradation of cIAP-2 not bound to TRAF2 relative to TRAF2-bound cIAP-2 and, if the Smac mimetic has such characteristic, then informing a healthcare treatment prescriber or a patient that a Smac mimetic with the structure of the so-assayed Smac mimetic has such characteristic.

In additional illustrative embodiments, the invention comprises any one or more of the above methods that further comprises administering a second cancer-related therapy, such as, e.g., radiation, chemotherapy, immunotherapy, photodynamic therapy, and combinations thereof.

In a further illustrative embodiment, the invention comprises a method of treating an autoimmune disease, in which the condition is caused or exacerbated by abnormal regulation of apoptosis, in a mammal in need thereof, including, for example, systemic lupus erythematosus, psoriasis, and idiopathic thrombocytopenic purpura (Morbus Werlhof) that comprises internally administering to the animal an effective amount of a Smac mimetic selected, at least in part, on the basis of its cIAP and TRAF2-bound cIAP degradation profile, as described in this specification.

In a further illustrative embodiment, the invention comprises a method of treating a benign proliferative disease that comprises internally administering to the animal an effective amount of a Smac mimetic selected, at least in part, on the basis of its cIAP and TRAF2-bound cIAP degradation profile, as described in this specification. Smac mimetics can be monovalent or bivalent although it appears that bivalent Smac mimetics are more effective.

In a further illustrative embodiment, the bivalent Smac mimetic of the invention has the cIAP degradation profile described herein and has the formula I, which follows:

[P1-P2-P3-P4]-L-[P1′-P2′-P3′-P4′]

wherein P1-P2-P3- and P1′-P2′-P3′-correspond to, i.e., mimic, the N-terminal Ala-Val-Pro- of mature Smac and P4 and P4′ correspond to Phe, Tyr, Ile, or Val and L is a linking group, or bond, covalently linking [P1-P2-P3-P4] to [P1′-P2′-P3′-P4′].

Excluded from the scope of compounds of this invention are previously described compounds known to have the cIAP degradation profile described herein. Thus, Compound 15 of US8283372 is excluded from the scope of compounds of this invention. Said Compound 15, which is birinapant, has the following structure:

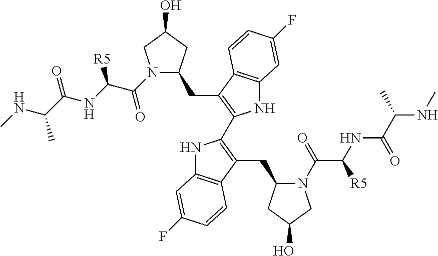

wherein R5 is —CH2CH3.

### BRIEF DESCRIPTION OF THE FIGURES

FIG. 1 shows results from a western blot analysis of green fluorescence fused cIAP-1 (FIG. 1A) and green fluorescence fused cIAP-2 (FIG. 1B) degradation as a result of treatment with a Smac mimetic having a cIAP degradation profile consistent with this invention. Western blot analysis of total lysates or TRAF-2 pull-down material. Cell line was treated with either compounds 5 or 15 at shown concentrations and lysates generated. The lysates were loaded and probed for proteins as shown. In the co-IP GFP cIAP1 lane, anti-TRAF-2 was used to pull-down TRAF-2; anti-cIAP 1 or anti-cIAP 2 antibody was used to detect for the presence of either cIAP1 or cIAP2 bound to TRAF2.

FIG. 2 is a western blot showing that Compound 15 removes cIAP-1 from the TRAF2 complex in HeLA cells as described in the Examples. Western blot analysis of total lysates or TRAF-2 pull-down material. Cell line was treated with either birinapant or GT14433 at shown concentrations and lysates generated. The lysates were loaded and probed for proteins as shown. In the TRAF2-IP lane, anti-TRAF-2 was used to pull-down TRAF-2; anti-cIAP1 antibody was used to detect for the presence of cIAP 1 bound to TRAF2.

FIG. 3 is a set of two charts showing quantification of cIAP-1 intensity in total lysate (A) and TRAF2-bound cIAP1 following treatment with Compound 15 as described in the Examples.

### DETAILED DESCRIPTION OF THE INVENTION

Inhibitor of Apoptosis Proteins (IAPB) regulate diverse extrinsic and intrinsic cellular apoptotic signals. Among the 8 human IAPB, cIAP-1, cIAP-2 and XIAP have recently been identified as the primary targets of small molecule Smac-mimetic compounds. Similar to endogenous Smac, Smac mimetics bind to the conserved BIR domains of the primary IAP target proteins and antagonize their anti-apoptotic functions. Smac mimetics have been shown to enhance the cytotoxicity of chemotherapeutic drugs as well as biologic agents (TNFα and TRAIL) in vitro and in vivo. Several small molecule Smac mimetics are currently in clinical testing as cancer therapeutics. Here we show that treatment of a proliferative disorder in a patient with a Smac mimetic, selected on the basis that it degrades cIAP-1 not bound to TRAF2, cIAP-1 bound to TRAF2, and cIAP2 bound to TRAF-2 but that does not substantially degrade cIAP-2 not bound to TRAF2 can be better tolerated (i.e., fewer adverse effects) than treatment with a Smac mimetic that degrades both cIAP-2 not bound to TRAF2, as well as TRAF2-bound cIAP-2 to substantially the same extent.

Smac mimetics used in the practice of the current invention can cause degradation of cIAP-2 not bound to TRAF2, but the extent of such degradation on a percentage basis will be less than the extent of degradation of TRAF2-bound cIAP-2. The significance of the difference in effects of a Smac mimetic on the cIAP-2 not bound to TRAF2, has been observed to correlate with the tolerability (or safety profile) of a Smac mimetic in animals. If a first Smac mimetic causes less degradation of cIAP-2 not bound to TRAF2, relative to degradation of TRAF2-bound cIAP-2, than a second Smac mimetic, i.e., a structurally different Smac mimetic, then the first Smac mimetic is likely to be better tolerated in (i.e., more safely administered to) animals. More specifically, a skilled person can select two Smac mimetics, each causing degradation of cIAP-1 not bound to TRAF2, TRAF2-bound cIAP-1 and TRAF2-bound cIAP-2 with one exhibiting a different (lesser) degree of degradation of cIAP-2 not bound to TRAF2, then the compound that causes less degradation of cIAP-2 not bound to TRAF2, is likely to be better tolerated with no significant loss in antitumor potency.

The degradation kinetics of non-TRAF2-bound cIAP-1, non-TRAF2-bound cIAP-2, TRAF2-bound cIAP-1 and TRAF2-bound cIAP-2 can be measured by western analysis, as described in the Examples, below. The extent of degradation can be observed visually in such assays over a period of time. For example, the extent of degradation of non-TRAF2-bound cIAP-2 and TRAF2-bound cIAP-2 may appear to be substantially the same immediately following treatment of cells with a Smac mimetic but after several minutes, e.g., after 15 to 30 minutes, increased degradation of TRAF2-bound cIAP-2 relative to degradation of non-TRAF2-bound cIAP-2 may be observed in treated cells. Differences in extent of degradation can also be quantified. For example, in the case of western analysis using green fluorescence protein tagged cIAPs, the extent of degradation can be quantified using a device that measures the intensity of fluorescence.

For a Smac mimetic that is likely to be better tolerated in animals, the extent of degradation of non-TRAF2-bound cIAP-2 will generally be less than 75% of (or about 75% of), i.e., about 75% or less than, the extent of degradation of TRAF2-bound cIAP-2 at relevant concentrations, for at least about 15 minutes, e.g., 30 to 120 minutes (or about 30 to about 120 minutes). The amount of Smac mimetic used in such assay will vary with the potency of the Smac mimetic but will generally be less than 1 uM, such as e.g., between about 1 and about 500 nM or between about 10 and about 150 nM

In some cases, the extent of degradation of non-TRAF2-bound cIAP-2 will be less than 50% of (or about 50% of), i.e., about 50% or less than; or less than 25% of (or about 25% of), i.e., about 25% or less than; or less than 10% of (or about 10% of), i.e., about 10% or less than, the extent of the degradation of TRAF2-bound cIAP-2. For example, in a cIAP degradation assay with a Smac mimetic having a cIAP degradation profile of the invention, TRAF2-bound cIAP-2 may be about 70-75% degraded after 30 minutes (i.e., only about 30% of the originally detected amount of TRAF2-bound cIAP-2 is still detectable); whereas non-TRAF2-bound cIAP-2 may only be about 35-40% degraded (i.e., 60% to 65% of the originally detected amount of non-TRAF2-bound cIAP-2 is still detectable) after 30 minutes. In this case, the Smac mimetic is said to degrade non-TRAF2-bound cIAP-2 at about 50% or less than the extent of degradation of TRAF2-bound cIAP-2 (35% to 40% divided by (70% to 75%)=about 50%).

The Smac mimetic that is assayed to determine its cIAP degradation profile may not have been determined to be a Smac mimetic prior to conduct of the assay. In any event, it is expected that such compound will require additional pre-clinical and clinical testing prior to seeking regulatory approval to administer to humans or other animals. It will also be understood that the Smac mimetic that is ultimately formulated, administered to subjects, submitted for regulatory approval, etc., is one that has as its active moiety the same chemical structure as the Smac mimetic that was assayed and selected, at least in part, on the basis of its cIAP degradation profile but that such Smac mimetic may also comprise pharmaceutically acceptable salts or solvates of such active moiety. For example, a certain compound, or a library of compounds, may be assayed to determine its cIAP degradation profile. A compound having the cIAP degradation profile that demonstrates degradation of cIAP-1 not bound to TRAF2, TRAF2-bound cIAP-1 and TRAF2-bound cIAP-2 and a lesser degree of degradation of cIAP-2 not bound to TRAF2, relative to TRAF2-bound cIAP-2 may then be selected and further analyzed to determine that the compound is a Smac mimetic. Such compound, whether previously or subsequently determined to be a Smac mimetic, may then be put through an extensive battery of pre-clinical tests and, if successful, clinical trials, to develop sufficient information and data to prepare a dossier for submission to a regulatory agency, e.g., the U.S. Food and Drug Administration. In the course of such testing, the compound may be formulated as a salt such that the drug product that is ultimately administered to a patient is a salt of the compound originally assayed to determine its cIAP degradation profile.

A Smac mimetic for use in the invention binds to and inhibits the activity of one or more IAPB, such as a cellular IAP (cIAP, e.g., cIAP-1 or cIAP-2) or X-linked IAP(XIAP). In some embodiments, the IAP antagonist preferentially binds XIAP, cIAP-1, or cIAP-2. A Smac mimetic is a mimetic or peptidomimetic of the N-terminal 4-amino acids of mature Smac (Ala-Val-Pro-Ile) or, more generally, Ala-Val-Pro-Xaa, wherein Xaa is Phe, Tyr, Ile, or Val, preferably Phe or Ile. Bivalent Smac mimetics have been shown to be more effective than monovalent Smac mimetics.

An illustrative genus of bivalent Smac mimetics has the generic structure of formula I, which follows:

[P1-P2-P3-P4]-L-[P1′-P2′-P3′-P4′]

wherein
P1 and P1′ are NHR¹—CHR²—C(O)—;
P2 and P2′ are —NH—CHR³—C(O)—;
P3 and P3′ are pyrrolidine, pyrrolidine fused to a cycloalkyl, or pyrrolidine fused to a heterocycloalkyl having a —N— heteroatom, and wherein the pyrrolidine of P3/P3′ is bound to P2/P2′ by an amide bond;
P4 and P4′ are -M-Qₚ-R⁷;
R¹ is —H or —CH3;
R² is —CH3 or —CH2CH3;
R³ is C2-6 alkyl, C2-6 alkoxy, cycloalkyl, heterocycloalkyl, aryl or heteroaryl, optionally substituted in each case;
M is a covalent bond, C1-6 alkylene, substituted C1-C6 alkylene such as but not limited to —C(O)—;
Q is a covalent bond, C1-6 alkylene, substituted C1-C6 alkylene, —O— or —NR⁸—, provided that M is not a covalent bond if: (1) -M- is bound directly to the 2 position of a P3/P3′ pyrrolidine or to a heteroatom in a P3/P3′ pyrrolidine-heterocycloalkyl bicycle and (2) Q is —O— or —NR⁸—;
p is 0 or 1;
R⁷ is cycloalkyl, cycloalkylaryl, aryl or heteroaryl, optionally substituted in each case;
R⁸ is —H or C1-6 alkyl;
L is a linking group, or bond, covalently linking [P1-P2-P3-P4] to [P1′-P2′-P3′-P4′]. For the avoidance of doubt, when Q is absent, i.e., p is 0, and M is a covalent bond, then R⁷ is bound directly to P3/P3′.

“Alkyl” (monovalent) and “alkylene” (divalent) when alone or as part of another term (e.g., alkoxy) mean branched or unbranched, saturated aliphatic hydrocarbon group, having up to 12 carbon atoms unless otherwise specified. Examples of particular alkyl groups include, but are not limited to, methyl, ethyl, n-propyl, isopropyl, n-butyl, iso-butyl, sec-butyl, tert-butyl, n-pentyl, 2-methylbutyl, 2,2-dimethylpropyl, n-hexyl, 2-methylpentyl, 2,2-dimethylbutyl, n-heptyl, 3-heptyl, 2-methylhexyl, and the like. The term, “lower,” when used to modify alkyl, alkenyl, etc., means 1 to 4 carbon atoms, branched or linear so that, e.g., the terms “lower alkyl”, “C₁-C₄ alkyl” and “alkyl of 1 to 4 carbon atoms” are synonymous and used interchangeably to mean methyl, ethyl, 1-propyl, isopropyl, 1-butyl, sec-butyl or t-butyl. Examples of alkylene groups include, but are not limited to, methylene, ethylene, n-propylene, n-butylene and 2-methyl-butylene.

The term substituted alkyl refers to alkyl moieties having substituents replacing one or more hydrogens on one or more (often no more than four) carbon atoms of the hydrocarbon backbone. Such substituents are independently selected from the group consisting of: a halogen (e.g., I, Br, Cl, or F, particularly fluoro(F)), hydroxy, amino, cyano, mercapto, alkoxy (such as a C₁-C₆ alkoxy, or a lower (C₁-C₄) alkoxy, e.g., methoxy or ethoxy to yield an alkoxyalkyl), aryloxy (such as phenoxy to yield an aryloxyalkyl), nitro, oxo (e.g., to form a carbonyl), carboxyl (which is actually the combination of an oxo and hydroxy substituent on a single carbon atom), carbamoyl (an aminocarbonyl such as NR₂C(O)—, which is the substitution of an oxo and an amino on a single carbon atom), cycloalkyl (e.g., a cycloalkylalkyl), aryl (resulting for example in aralkyls such as benzyl or phenylethyl), heterocyclylalkyl (e.g., heterocycloalkylalkyl), heteroaryl (e.g., heteroarylalkyl), alkylsulfonyl (including lower alkylsulfonyl such as methylsulfonyl), arylsulfonyl (such as phenylsulfonyl), and —OCF₃ (which is a halogen substituted alkoxy). The invention further contemplates that several of these alkyl substituents, including specifically alkoxy, cycloalkyl, aryl, heterocyclyalkyl and heteroaryl, are optionally further substituted as defined in connection with each of their respective definitions provided below. In addition, certain alkyl substituent moieties result from a combination of such substitutions on a single carbon atom. For example, an ester moiety, e.g., an alkoxycarbonyl such as methoxycarbonyl, or tert-butoxycarbonyl (Boc) results from such substitution. In particular, methoxycarbonyl and Boc are substituted alkyls that result from the substitution on a methyl group (—CH₃) of both an oxo (═O) and an unsubstituted alkoxy, e.g., a methoxy (CH₃—O) or a tert-butoxy ((CH₃)₃C—O—), respectively replacing the three hydrogens. Similarly, an amide moiety, e.g., an alkylaminocarbonyl, such as dimethlyaminocarbonyl or methylaminocarbonyl, is a substituted alkyl that results from the substitution on a methyl group (—CH₃) of both an oxo (═O) and a mono-unsubstitutedalkylamino or, diunsubstitutedalkylamino, e.g., dimethylamino (—N—(CH₃)₂), or methylamino (—NH—(CH₃)) replacing the three hydrogens (similarly an arylaminocarbonyl such as diphenylaminocarbonyl is a substituted alkyl that results from the substitution on a methyl group (—CH₃) of both an oxo (═O) and a mono-unsubstitutedaryl(phenyl)amino). Exemplary substituted alkyl groups further include cyanomethyl, nitromethyl, hydroxyalkyls such as hydroxymethyl, trityloxymethyl, propionyloxymethyl, amino alkyls such as aminomethyl, carboxylalkyls such as carboxymethyl, carboxyethyl, carboxypropyl, 2,3-dichloropentyl, 3-hydroxy-5-carboxyhexyl, acetyl (e.g., an alkanoyl, where in the case of acetyl the two hydrogen atoms on the —CH₂ portion of an ethyl group are replaced by an oxo (═O)), 2-aminopropyl, pentachlorobutyl, trifluoromethyl, methoxyethyl, 3-hydroxypentyl, 4-chlorobutyl, 1,2-dimethyl-propyl, pentafluoroethyl, alkyloxycarbonylmethyl, allyloxycarbonylaminomethyl, carbamoyloxymethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichloro (n-butyl), 2-amino (iso-propyl), cycloalkylcarbonyl (e.g., cuclopropylcarbonyl) and 2-carbamoyloxyethyl. Particular substituted alkyls are substituted methyl groups. Examples of substituted methyl group include groups such as hydroxymethyl, protected hydroxymethyl (e.g., tetrahydropyranyl-oxymethyl), acetoxymethyl, carbamoyloxymethyl, trifluoromethyl, chloromethyl, carboxymethyl, carboxyl (where the three hydrogen atoms on the methyl are replaced, two of the hydrogens are replaced by an oxo (═O) and the other hydrogen is replaced by a hydroxy (—OH)), tert-butoxycarbonyl (where the three hydrogen atoms on the methyl are replaced, two of the hydrogens are replaced by an oxo (═O) and the other hydrogen is replaced by a tert-butoxy (—O—C(CH₃)₃), bromomethyl and iodomethyl. When the specification and especially the claims refer to a particular substituent for an alkyl, that substituent can potentially occupy one or more of the substitutable positions on the allyl. For example, reciting that an alkyl has a fluoro substituent, would embrace mono-, di-, and possibly a higher degree of substitution on the alkyl moiety.

The term substituted alkylene refers to alkylene moieties having substituents replacing one or more hydrogens on one or more (often no more than four) carbon atoms of the hydrocarbon backbone where the alkylene is similarly substituted with groups as set forth above for alkyl.

Alkoxy is —O-alkyl. A substituted alkoxy is —O-substituted alkyl, where the alkoxy is similarly substituted with groups as set forth above for alkyl. One substituted alkoxy is acetoxy where two of the hydrogens in ethoxy (e.g., —O—CH₂—CH₃) are replaced by an oxo, (═O) to yield —O—C(O)—CH₃; another is an aralkoxy where one of the hydrogens in the alkoxy is replaced by an aryl, such as benzyloxy, and another is a carbamate where two of the hydrogens on methoxy (e.g., —O—CH₃) are replaced by oxo (═O) and the other hydrogen is replaced by an amino (e.g., —NH₂, —NHR or —NRR) to yield, for example, —O—C(O)—NH₂. A lower alkoxy is —O-lower alkyl.

“Alkenyl” (monovalent) and “alkenylene” (divalent) when alone or as part of another term mean an unsaturated hydrocarbon group containing at least one carbon-carbon double bond, typically 1 or 2 carbon-carbon double bonds, which may be linear or branched and which have at least 2 and up to 12 carbon atoms unless otherwise specified. Representative alkenyl groups include, by way of example, vinyl, allyl, isopropenyl, but-2-enyl, n-pent-2-enyl, and n-hex-2-enyl.

The terms substuituted alkenyl and substituted alkenylene refer to alkenyl and alkenylene moieties having substituents replacing one or more hydrogens on one or more (often no more than four) carbon atoms of the hydrocarbon backbone. Such substituents are independently selected from the group consisting of: halo (e.g., I, Br, Cl, F), hydroxy, amino, cyano, alkoxy (such as C₁-C₆ alkoxy), aryloxy (such as phenoxy), nitro, mercapto, carboxyl, oxo, carbamoyl, cycloalkyl, aryl, heterocyclyl, heteroaryl, alkylsulfonyl, arylsulfonyl and —OCF₃.

“Alkynyl” means a monovalent unsaturated hydrocarbon group containing at least one carbon-carbon triple bond, typically 1 carbon-carbon triple bond, which may be linear or branched and which have at least 2 and up to 12 carbon atoms unless otherwise specified. Representative alkynyl groups include, by way of example, ethynyl, propargyl, and but-2-ynyl.

“Cycloalkyl” when alone or as part of another term means a saturated or partially unsaturated cyclic aliphatic hydrocarbon group (carbocycle group), having 3 to 8 carbon atoms unless otherwise specified, such as cyclopropyl, cyclobutyl, cyclopentyl and cyclohexyl, and further includes polycyclic, including fused cycloalkyls such as 1,2,3,4-tetrahydronaphthalenyls (1,2,3,4-tetrahydronaphthalen-1-yl, and 1,2,3,4-tetrahydronaphthalen-2-yl), indanyls (indan-lyl, and indan-2-yl), isoindenyls (isoinden-1-yl, isoinden-2-yl, and isoinden-3-yl) and indenyls (inden-1-yl, inden-2-yl and inden-3-yl). A lower cycloalkyl has from 3 to 6 carbon atoms and includes cyclopropyl, cyclobutyl, cyclopentyl and cyclohexyl.

The term substituted cycloalkyl refers to cycloalkyl moieties having substituents replacing one or more hydrogens on one or more (often no more than four) carbon atoms of the hydrocarbon backbone. Such substituents are independently selected from the group consisting of: halo (e.g., I, Br, Cl, F), hydroxy, amino, cyano, alkoxy (such as C₁-C₆ alkoxy), substituted alkoxy, aryloxy (such as phenoxy), nitro, mercapto, carboxyl, oxo, carbamoyl, alkyl, substituted alkyls such as trifluoromethyl, aryl, substituted aryls, heterocyclyl, heteroaryl, alkylsulfonyl, arylsulfonyl and —OCF₃. When the specification and especially the claims refer to a particular substuituent for a cycloalkyl, that substituent can potentially occupy one or more of the substitutable positions on the cycloalkyl. For example, reciting that a cycloalkyl has a fluoro substituent, would embrace mono-, di-, and a higher degree of substitution on the cycloalkyl moiety. Examples of cycloalkyls include cyclopropy, cyclobutyl, cyclopentyl, cyclohexyl, tetrahydronaphthyl and indanyl.

“Aryl” when used alone or as part of another term means an aromatic carbocyclic group whether or not fused having the number of carbon atoms designated, or if no number is designated, from 6 up to 14 carbon atoms. Particular aryl groups include phenyl, naphthyl, biphenyl, phenanthrenyl, naphthacenyl, indolyl, and the like (see e.g. Lang's Handbook of Chemistry (Dean, J. A., ed) 13^{th} ed. Table 7-2 [1985]).

The term substituted aryl refers to aryl moieties having substituents replacing one or more hydrogens on one or more (usually no more than six) carbon atoms of the aromatic hydrocarbon core. Such substituents are independently selected from the group consisting of: halo (e.g., I, Br, Cl, F), hydroxy, amino, cyano, alkoxy (such as C₁-C₆ alkoxy and particularly lower alkoxy), substituted alkoxy, aryloxy (such as phenoxy), nitro, mercapto, carboxyl, carbamoyl, alkyl, substituted alkyl (such as trifluoromethyl), aryl, —OCF₃, alkylsulfonyl (including lower alkylsulfonyl), arylsulfonyl, heterocyclyl and heteroaryl. Examples of such substituted phenyls include but are not limited to a mono- or di (halo) phenyl group such as 2-chlorophenyl, 2-bromophenyl, 4-chlorophenyl, 2,6-dichlorophenyl, 2,5-dichlorophenyl, 3,4-dichlorophenyl, 3-chlorophenyl, 3-bromophenyl, 4-bromophenyl, 3,4-dibromophenyl, 3-chloro-4-fluorophenyl, 2-fluorophenyl; 3-fluorophenyl, 4-fluorophenyl, a mono- or di(hydroxy)phenyl group such as 4-hydroxyphenyl, 3-hydroxyphenyl, 2,4-dihydroxyphenyl, the protected-hydroxy derivatives thereof; a nitrophenyl group such as 3- or 4-nitrophenyl; a cyanophenyl group, for example, 4-cyanophenyl; a mono- or di(lower alkyl)phenyl group such as 4-methylphenyl, 2,4-dimethylphenyl, 2-methylphenyl, 4-(iso-propyl)phenyl, 4-ethylphenyl, 3-(n-propyl)phenyl; a mono or di(alkoxy)phenyl group, for example, 3,4-dimethoxyphenyl, 3-methoxy-4-benzyloxyphenyl, 3-methoxy-4-(1-chloromethyl)benzyloxy-phenyl, 3-ethoxyphenyl, 4-(isopropoxy)phenyl, 4-(t-butoxy)phenyl, 3-ethoxy-4-methoxyphenyl; 3- or 4-trifluoromethylphenyl; a mono- or dicarboxyphenyl or (protected carboxy)phenyl group such 4-carboxyphenyl; a mono- or di(hydroxymethyl)phenyl or (protected hydroxymethyl)phenyl such as 3-(protected hydroxymethyl)phenyl or 3,4-di(hydroxymethyl)phenyl; a mono- or di(aminomethyl)phenyl or (protected aminomethyl)phenyl such as 2-(aminomethyl)phenyl or 2,4-(protected aminomethyl)phenyl; or a mono- or di(N-(methylsulfonylamino))phenyl such as 3-(N-methylsulfonylamino)phenyl. Also, the substituents, such as in a disubstituted phenyl groups, can be the same or different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl, as well as for trisubstituted phenyl groups where the substituents are different, as for example 3-methoxy-4-benzyloxy-6-methyl sulfonylamino, 3-methoxy-4-benzyloxy-6-phenyl sulfonylamino, and tetrasubstituted phenyl groups where the substituents are different such as 3-methoxy-4-benzyloxy-5-methyl-6-phenyl sulfonylamino. Particular substituted phenyl groups are 2-chlorophenyl, 2-aminophenyl, 2-bromophenyl, 3-methoxyphenyl, 3-ethoxy-phenyl, 4-benzyloxyphenyl, 4-methoxyphenyl, 3-ethoxy-4-benzyloxyphenyl, 3,4-diethoxyphenyl, 3-methoxy-4-benzyloxyphenyl, 3-methoxy-4-(1-chloromethyl)benzyloxy-phenyl, 3-methoxy-4-(1-chloromethyl)benzyloxy-6-methyl sulfonyl aminophenyl groups. When the specification and especially the claims refer to a particular substituent for an aryl, that substituent can potentially occupy one or more of the substitutable positions on the aryl. For example, reciting that an aryl has a fluoro substituent, would embrace mono-, di-, tri, tetra and a higher degree of substitution on the aryl moiety. Fused aryl rings may also be substituted with the substituents specified herein, for example with 1, 2 or 3 substituents, in the same manner as substituted alkyl groups. The terms aryl and substituted aryl do not include moieties in which an aromatic ring is fused to a saturated or partially unsaturated aliphatic ring.

Aryloxy is —O-aryl. A substituted aryloxy is —O-substituted aryl, where the suitable substituents are those described for a substituted aryl.

“Heterocyclic group”, “heterocyclic”, “heterocycle”, “heterocyclyl”, “heterocycloalkyl” or “heterocyclo” alone and when used as a moiety in a complex group, are used interchangeably and refer to any mono-, bi-, or tricyclic, saturated or unsaturated, non-aromatic hetero-atom-containing ring system having the number of atoms designated, or if no number is specifically designated then from 5 to about 14 atoms, where the ring atoms are carbon and at least one heteroatom and usually not more than four heteroatoms (i.e., nitrogen, sulfur or oxygen). Included in the definition are any bicyclic groups where any of the above heterocyclic rings are fused to an aromatic ring (i.e., an aryl (e.g., benzene) or a heteroaryl ring). In a particular embodiment the group incorporates 1 to 4 heteroatoms. Typically, a 5-membered ring has 0 to 1 double bonds and a 6- or 7-membered ring has 0 to 2 double bonds and the nitrogen or sulfur heteroatoms may optionally be oxidized (e.g. SO, SO₂), and any nitrogen heteroatom may optionally be quaternized. Particular unsubstituted non-aromatic heterocycles include morpholinyl (morpholino), pyrrolidinyls, oxiranyl, indolinyls, 2,3-dihydroindolyl, isoindolinyls, 2,3-dihydroisoindolyl, tetrahydroquinolinyls, tetrahydroisoquinolinyls, oxetanyl, tetrahydrofuranyls, 2,3-dihydrofuranyl, 2H-pyranyls, tetrahydropyranyls, aziridinyls, azetidinyls, 1-methyl-2-pyrrolyl, piperazinyls and piperidinyls.

The term substituted heterocyclo refers to heterocyclo moieties having substituents replacing one or more hydrogens on one or more (usually no more than six) atoms of the heterocyclo backbone. Such substituents are independently selected from the group consisting of: halo (e.g., I, Br, Cl, F), hydroxy, amino, cyano, alkoxy (such as C₁-C₆ alkoxy), substituted alkoxy, aryloxy (such as phenoxy), nitro, carboxyl, oxo, carbamoyl, alkyl, substituted alkyl (such as trifluoromethyl), —OCF₃, aryl, substituted aryl, alkylsulfonyl (including lower alkylsulfonyl), and arylsulfonyl. When the specification and especially the claims refer to a particular substuituent for a heterocycloalkyl, that substituent can potentially occupy one or more of the substitutable positions on the heterocycloalkyl. For example, reciting that a heterocycloalkyl has a fluoro substituent, would embrace mono-, di-, tri, tetra and a higher degree of substitution on the heterocycloalkyl moiety.

“Heteroaryl” alone and when used as a moiety in a complex group refers to any mono-, bi-, or tricyclic aromatic ring system having the number of atoms designated, or if no number is specifically designated then at least one ring is a 5-, 6- or 7-membered ring and the total number of atoms is from 5 to about 14 and containing from one to four heteroatoms selected from the group consisting of nitrogen, oxygen, and sulfur (Lang's Handbook of Chemistry, supra). Included in the definition are any bicyclic groups where any of the above heteroaryl rings are fused to a benzene ring. The following ring systems are examples of the heteroaryl groups denoted by the term “heteroaryl”: thienyls (alternatively called thiophenyl), furyls, imidazolyls, pyrazolyls, thiazolyls, isothiazolyls, oxazolyls, isoxazolyls, triazolyls, thiadiazolyls, oxadiazolyls, tetrazolyls, thiatriazolyls, oxatriazolyls, pyridyls, pyrimidinyls (e.g., pyrimidin-2-yl), pyrazinyls, pyridazinyls, thiazinyls, oxazinyls, triazinyls, thiadiazinyls, oxadiazinyls, dithiazinyls, dioxazinyls, oxathiazinyls, tetrazinyls, thiatriazinyls, oxatriazinyls, dithiadiazinyls, imidazolinyls, dihydropyrimidyls, tetrahydropyrimidyls, tetrazolo[1,5-b]pyridazinyl and purinyls, as well as benzo-fused derivatives, for example benzoxazolyls, benzofuryls, benzothienyls, benzothiazolyls, benzothiadiazolyl, benzotriazolyls, benzoimidazolyls, isoindolyls, indazolyls, indolizinyls, indolyls, naphthyridines, pyridopyrimidines, phthalazinyls, quinolyls, isoquinolyls and quinazolinyls.

The term substituted heteroaryl refers to heteroaryl moieties (such as those identified above) having substituents replacing one or more hydrogens on one or more (usually no more than six) atoms of the heteroaryl backbone. Such substituents are independently selected from the group consisting of: halo (e.g., I, Br, Cl, F), hydroxy, amino, cyano, alkoxy (such as C1-C6 alkoxy), aryloxy (such as phenoxy), nitro, mercapto, carboxyl, carbamoyl, alkyl, substituted alkyl (such as trifluoromethyl), —OCF₃, aryl, substituted aryl, alkylsulfonyl (including lower alkylsulfonyl), and arylsulfonyl. When the specification and especially the claims refer to a particular substuituent for a heteroaryl, that substituent can potentially occupy one or more of the substitutable positions on the heteroaryl. For example, reciting that a heteroaryl has a fluoro substituent, would embrace mono-, di-, tri, tetra and a higher degree of substitution on the heteroaryl moiety.

Particular “heteroaryls” (including “substituted heteroaryls”) include; 1H-pyrrolo[2,3-b]pyridine, 1,3-thiazol-2-yl, 4-(carboxymethyl)-5-methyl-1,3-thiazol-2-yl, 1,2,4-thiadiazol-5-yl, 3-methyl-1,2,4-thiadiazol-5-yl, 1,3,4-triazol-5-yl, 2-methyl-1,3,4-triazol-5-yl, 2-hydroxy-1,3,4-triazol-5-yl, 2-carboxy-4-methyl-1,3,4-triazol-5-yl, 1,3-oxazol-2-yl, 1,3,4-oxadiazol-5-yl, 2-methyl-1,3,4-oxadiazol-5-yl, 2-(hydroxymethyl)-1,3,4-oxadiazol-5-yl, 1,2,4-oxadiazol-5-yl, 1,3,4-thiadiazol-5-yl, 2-thiol-1,3,4-thiadiazol-5-yl, 2-(methylthio)-1,3,4-thiadiazol-5-yl, 2-amino-1,3,4-thiadiazol-5-yl, 1H-tetrazol-5-yl, 1-methyl-1H-tetrazol-5-yl, 1-(1-(dimethylamino) eth-2-yl)-1H-tetrazol-5-yl, 1-(carboxymethyl)-1H-tetrazol-5-yl, 1-(methylsulfonic acid)-1H-tetrazol-5-yl, 2-methyl-1H-tetrazol-5-yl, 1,2,3-triazol-5-yl, 1-methyl-1,2,3-triazol-5-yl, 2-methyl-1,2,3-triazol-5-yl, 4-methyl-1,2,3-triazol-5-yl, pyrid-2-yl N-oxide, 6-methoxy-2-(n-oxide)-pyridaz-3-yl, 6-hydroxypyridaz-3-yl, 1-methylpyrid-2-yl, 1-methylpyrid-4-yl, 2-hydroxypyrimid-4-yl, 1,4,5,6-tetrahydro-5,6-dioxo-4-methyl-as-triazin-3-yl, 1,4,5,6-tetrahydro-4-(formylmethyl)-5,6-dioxo-as-triazin-3-yl, 2,5-dihydro-5-oxo-6-hydroxy-astriazin-3-yl, 2,5-dihydro-5-oxo-6-hydroxy-as-triazin-3-yl, 2,5-dihydro-5-oxo-6-hydroxy-2-methyl-astriazin-3-yl, 2,5-dihydro-5-oxo-6-hydroxy-2-methyl-as-triazin-3-yl, 2,5-dihydro-5-oxo-6-methoxy-2-methyl-as-triazin-3-yl, 2,5-dihydro-5-oxo-as-triazin-3-yl, 2,5-dihydro-5-oxo-2-methyl-as-triazin-3-yl, 2,5-dihydro-5-oxo-2,6-dimethyl-as-triazin-3-yl, tetrazolo[1, 5-b]pyridazin-6-yl, 8-aminotetrazolo[1,5-b]-pyridazin-6-yl, quinol-2-yl, quinol-3-yl, quinol-4-yl, quinol-5-yl, quinol-6-yl, quinol-8-yl, 2-methyl-quinol-4-yl, 6-fluoro-quinol-4-yl, 2-methyl,8-fluoro-quinol-4-yl, isoquinol-5-yl, isoquinol-8-yl, isoquinol-1-yl, and quinazolin-4-yl. An alternative group of “heteroaryl” includes: 5-methyl-2-phenyl-2H-pyrazol-3-yl, 4-(carboxymethyl)-5-methyl-1,3-thiazol-2-yl, 1,3,4-triazol-5-yl, 2-methyl-1,3,4-triazol-5-yl, 1H-tetrazol-5-yl, 1-methyl-1H-tetrazol-5-yl, 1-(1-(dimethylamino) eth-2-yl)-1H-tetrazol-5-yl, 1-(carboxymethyl)-1H-tetrazol-5-yl, 1-(methylsulfonic acid)-1H-tetrazol-5-yl, 1,2,3-triazol-5-yl, 1,4,5,6-tetrahydro-5,6-dioxo-4-methyl-as-triazin-3-yl, 1,4,5,6-tetrahydro-4-(2-formylmethyl)-5,6-dioxo-as-triazin-3-yl, 2,5-dihydro-5-oxo-6-hydroxy-2-methyl-as-triazin-3-yl, 2,5-dihydro-5-oxo-6-hydroxy-2-methyl-as-triazin-3-yl, tetrazolo[1,5-b]pyridazin-6-yl, and 8-aminotetrazolo[1,5-b]pyridazin-6-yl.

L is a linking group or bond covalently linking one monomer, [P1-P2-P3-P4] to the other monomer, [P1′-P2′-P3′-P4′]. Commonly, -L-links P2 to P2′ position such as at R³ or P4 to P4′ such as at M, G, Q, or R⁷, or both P2 to P2′ and P4 to P4′. Illustrative examples of L are a single or double covalent bond, C1-12 alkylene, substituted C1-12 alkylene, C1-12 alkenylene, substituted C1-12 alkenylene, C1-12 alkynylene, substituted C1-12 alkynylene, Xₙ-phenyl-Yₙ, or Xₙ-(phenyl)₂—Yₙ, wherein X and Y are independently C1-6 alkylene, substituted C1-6 alkylene, C1-6 alkenylene, substituted C1-6 alkenylene, C1-6 alkynylene, substituted C1-6 alkynylene, or S(O)₂.

Illustrative P3/P3′ groups include, without limitation:

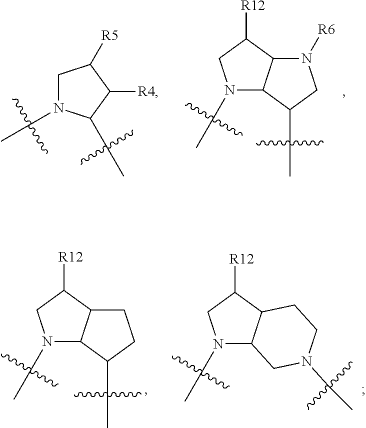

wherein R⁶ is —H, C1-6 alkyl, substituted C1-6 alkyl, C1-6 alkoxy, substituted C1-6 alkoxy, C1-6 alkylsulfonyl, arylsulfonyl, cycloalkyl, substituted cycloalkyl, heterocycloalkyl, substituted heterocycloalkyl, aryl, substituted aryl, heteroaryl, or substituted heteroaryl;
R⁴, R⁵, and R¹² are, independently, —H, —OH, C1-6 alkyl, C1-6 heteroalkyl, C1-6 alkoxy, aryloxy, cycloalkyl, heterocycloalkyl, aryl, C1-6 alkyl aryl, or heteroaryl, or C1-6 alkyl heteroaryl, optionally substituted in each case except when R⁴ is —H or —OH

An illustrative subgenus of Smac mimetics comprises compounds of formula I, above, wherein:
R¹ is —CH3;
R² is —CH3 or —CH2CH3;
P3 is

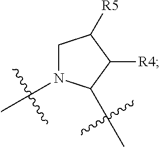

R³ is —C2-6 alkyl, C2-6 alkoxy, or cycloalkyl;
R⁴ is —H;
R⁵ is —H, —OH, C1-6 alkyl, or C1-6 alkoxy;
M is a single covalent bond, C1-6 alkylene, or substituted C1-6 alkylene; Q is a single covalent bond, C1-6 alkylene, substituted C1-6 alkylene, or NR¹⁸, provided that M is not a covalent bond if Q is —O— or —NR⁸;
n is 0 or 1;
R⁷ is aryl or heteroaryl;
R⁸ is —H or C1-6 alkyl;
L is a single or double covalent bond or is C1-4 alkylene, C1-4 alkenylene, or C1-4 alkynylene linking P4 to P4′ through M, Q, or R⁷.

In accordance with this invention, a Smac mimetic, selected in part on the basis of its relative potency against cIAP-1 and cIAP-2 not bound to TRAF2, TRAF2-bound cIAP-1 and TRAF2-bound cIAP-2, are used in the treatment of proliferative disorders, e.g.: various benign tumors or malignant tumors (cancer), benign proliferative diseases (e.g., psoriasis, benign prostatic hypertrophy, and restenosis), or autoimmune diseases (e.g., autoimmune proliferative glomerulonephritis, lymphoproliferative autoimmune responses).

Some embodiments of the invention include inducing apoptosis of cells, particularly pathologically proliferating cells. The methods can be carried out in vitro or in vivo.

The methods of the invention can include administration of such Smac mimetic in a combination therapy, e.g., in which such Smac mimetic is administered as part of a therapeutic regimen that also includes one or more additional active pharmaceutical ingredients, e.g., one or more additional biological or chemotherapeutic agents. Administration of multiple agents can be simultaneous or sequential. The Smac mimetic, selected for use in the present invention based on its cIAP degradation profile, is formulated into a pharmaceutical composition prior to administration to a patient. Such pharmaceutical compositions typically can be administered in the conventional manner by routes including systemic, subcutaneous, topical, or oral routes. Administration may be by intravenous injection, either as a bolus or infusion, but other routes of administration, including, among others, subcutaneous or oral administration, are not precluded. An intravenous formulation can contain, e.g., from 1 mg/mL up to and including 5 mg/mL of Compound 15 (U.S. Pat. No. 8,283,372) in sterile 0.05M citrate buffered PBS, pH5. Formulation may be by immediate release or prolonged release (e.g. pegylated formulation). Specific modes of administration and formulation will depend on the indication and other factors including the particular compound being administered. The amount of compound to be administered is that amount which is therapeutically effective, i.e., the amount that ameliorates the disease symptoms, i.e., that slows cancer progression or causes regression, without serious adverse effects relative to the disease being treated. An effective dose is one that over the course of therapy, which may be, e.g., 1 or more weeks, e.g., multiple courses of 3 weeks on/1 week off, results in treatment of the proliferative disorder, i.e., a decrease in the rate of disease progression, termination of disease progression, or regression or remission.

The dosage to be administered will depend on the potency and tolerability of the Smac mimetic and on other factors such as the characteristics of the subject being treated, e.g., the particular patient treated, age, weight, health, types of concurrent treatment, if any. Frequency of treatments can be easily determined by one of skill in the art (e.g., by the clinician).

For example, although Compound 15 is not among the compounds of the invention, information concerning treatment with this compound is provided to provide guidance relative to the administration of other compounds of similar potency and having a similar IAP antagonism profile.

Compound 15 can be administered by intravenous infusion at a dose of 0.1 to 100 mg/m² of patient body surface area (BSA), 1 to 80, 2 to 80, 2 to 65, 5 to 65, 10 to 65, 20 to 65, 30 to 65, 30 or >30 to 80, 30 or >30 to 65, 30 or >30 to 60, 30 or >30 to 55, or 30 or >30 to 50 mg/m², by intravenous infusion over an infusion period of about 1 to about 120 minutes, e.g., about 30 minutes. An illustrative dosing regimen for Compound 15 is one 30 minute infusion/week for 2 to 4, e.g., 2 or 3, consecutive weeks, followed by a week off. Such treatment cycle of two, three or four weeks on and one week off can be continued for as long as it is effective and tolerated. The dose is the maximum tolerated dose, with duration of treatment to continue until disease progression or unacceptable toxicity. Lower doses and less frequent schedule of administration would also be acceptable in combination with another therapy(ies).

Pharmaceutical compositions to be used comprise a therapeutically effective amount of a compound as described above, or a pharmaceutically acceptable salt or other form thereof together with one or more pharmaceutically acceptable excipients. The phrase “pharmaceutical composition” refers to a composition suitable for administration in medical or veterinary use. It should be appreciated that the determinations of proper dosage forms, dosage amounts, and routes of administration for a particular patient are within the level of ordinary skill in the pharmaceutical and medical arts.

Compositions suitable for parenteral administration conveniently comprise a sterile aqueous preparation of a compound or composition of the invention, which is preferably isotonic with the blood of the recipient. This aqueous preparation may be formulated according to known methods using suitable dispersing or wetting agents, emulsifying and suspending agents. Various antibacterial and antifungal agents, for example, parabens, chlorobutanol, phenol, and sorbic acid also may be included. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Prolonged absorption of the injectable pharmaceutical form can be brought about by the use of agents delaying absorption, for example, aluminum monostearate and gelatin. Carrier formulation suitable for subcutaneous, intravenous, intramuscular, etc. administrations can be found in Remington's Pharmaceutical Sciences, Mack Publishing Co., Easton, Pa. which is incorporated herein in its entirety by reference thereto.

A pharmaceutical composition in intravenous unit dose form may comprise, e.g., a vial or pre-filled syringe, or an infusion bag or device, each comprising an effective amount or a convenient fraction of an effective amount such that the contents of one vial or syringe are administered at a time.

Administration can be repeated up to about 4 times per day over a period of time, if necessary to achieve a cumulative effective dose, e.g., a cumulative dose effective to produce tumor stasis or regression. A dosing regimen can be, e.g., daily or twice-weekly intravenous injections, or, e.g., once weekly injections in cycles of three weeks on and one week off for as long as the treatment is effective, e.g., until disease progresses or the drug is not tolerated. The effective dose administered in each injection is an amount that is effective and tolerated.

An effective dose is one that over the course of therapy, which may be, e.g., 1 or more weeks, e.g., multiple courses of 3 weeks on/1 week off, results in treatment of the proliferative disorder, i.e., a decrease in the rate of disease progression, termination of disease progression, or regression or remission.

Solid dosage forms for oral administration include capsules, tablets, pills, powders, and granules. In such solid dosage forms, the compound is admixed with at least one inert pharmaceutically acceptable excipient such as (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents. Solid dosage forms such as tablets, dragees, capsules, pills, and granules also can be prepared with coatings and shells, such as enteric coatings and others well known in the art. The solid dosage form also may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients. Such solid dosage forms may generally contain from 1% to 95% (w/w) of the active compound. In certain embodiments, the active compound ranges from 5% to 70% (w/w).

Since one aspect of the present invention contemplates the treatment of the disease/conditions with a combination of pharmaceutically active agents that may be administered separately, the invention further relates to combining separate pharmaceutical compositions in kit form. The kit comprises two separate pharmaceutical compositions: one contains the Smac mimetic used in the method of the present invention, and a second one contains a second active pharmaceutical ingredient. The kit comprises a container for containing the separate compositions such as a divided bottle or a divided foil packet. Additional examples of containers include syringes, e.g., pre-filled syringes, boxes and bags. Typically, the kit comprises directions for the use of the separate components. The kit form is particularly advantageous when the separate components are preferably administered in different dosage forms (e.g., oral and parenteral), are administered at different dosage intervals, or when titration of the individual components of the combination is desired by the prescribing physician or veterinarian.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a substance of the present invention can consist of one tablet or capsule, while a daily dose of the second substance can consist of several tablets or capsules and vice versa. The memory aid should reflect this and aid in correct administration of the active agents.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

Liquid dosage forms for oral administration include pharmaceutically acceptable emulsions, solutions, suspensions, syrups, and elixirs. In addition to the compound or composition, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances. Besides such inert diluents, the composition can also include adjuvants, such as wetting agents, emulsifying and suspending agents, sweetening, flavoring, and perfuming agents.

The compounds and compositions used in the method of the present invention also may benefit from a variety of delivery systems, including time-released, delayed release or sustained release delivery systems. Such option may be particularly beneficial when the compounds and composition are used in conjunction with other treatment protocols as described in more detail below.

Many types of controlled release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active compound is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be desirable. Long-term release, as used herein, means that the implant is constructed and arranged to deliver therapeutic levels of the active compound for at least 30 days, and preferably 60 days. Long-term sustained release implants are well-known to those of ordinary skill in the art and include some of the release systems described above.

The compounds used in the method of the present invention and pharmaceutical compositions comprising compounds used in the method of the present invention can be administered to a subject suffering from cancer, an autoimmune disease or another disorder where a defect in apoptosis is implicated. In connection with such treatments, the patient can be treated prophylactically, acutely, or chronically using the compounds and compositions used in connection with the method of the present invention, depending on the nature of the disease. Typically, the host or subject in each of these methods is human, although other mammals may also benefit from the present invention.

A Smac mimetic selected on the basis of its cIAP degradation profile in accordance with this invention can be used for the treatment of cancer types which fail to undergo apoptosis. Thus, compounds used on the method of the present invention can be used to provide a therapeutic approach to the treatment of many kinds of solid tumors, including but not limited to carcinomas, sarcomas including Kaposi's sarcoma, erythroblastoma, glioblastoma, meningioma, astrocytoma, melanoma and myoblastoma. Treatment or prevention of non-solid tumor cancers such as leukemia is also contemplated by this invention. Indications may include, but are not limited to brain cancers, skin cancers, bladder cancers, ovarian cancers, breast cancers, gastric cancers, pancreatic cancers, colon cancers, blood cancers, lung cancers and bone cancers. Examples of such cancer types include neuroblastoma, intestine carcinoma such as rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, craniopharyngeoma, osteosarcoma, chondrosarcoma, myosarcoma, liposarcoma, fibrosarcoma, Ewing sarcoma and plasmocytoma.

Smac mimetics suitable for use in the method of the present invention will be active for treating human malignancies including, but not limited to, such human malignancies in which cIAP-1 and cIAP-2 are over-expressed (e.g., lung cancers, see Dai et al, Hu. Molec. Genetics, 2003 v 12 pp 791-801; leukemias (multiple references), and other cancers (Tamm et al, Clin Cancer Res, 2000, v 6, 1796-1803). Such IAP antagonists will be active in disorders that may be driven by inflammatory cytokines such as TNFα playing a pro-survival role (for example, there is a well defined role for TNFα acting as a survival factor in ovarian carcinoma, similarly for gastric cancers (see Kulbe, et al, Cancer Res 2007, 67, 585-592).

In addition to apoptosis defects found in tumors, defects in the ability to eliminate self-reactive cells of the immune system due to apoptosis resistance are considered to play a key role in the pathogenesis of autoimmune diseases. Autoimmune diseases are characterized in that the cells of the immune system produce antibodies against its own organs and molecules or directly attack tissues resulting in the destruction of the latter. A failure of those self-reactive cells to undergo apoptosis leads to the manifestation of the disease. Defects in apoptosis regulation have been identified in autoimmune diseases such as systemic lupus erythematosus or rheumatoid arthritis.

Examples of such autoimmune diseases include collagen diseases such as rheumatoid arthritis, systemic lupus erythematosus, Sharp's syndrome, CREST syndrome (calcinosis, Raynaud's syndrome, esophageal dysmotility, telangiectasia), dermatomyositis, vasculitis (Morbus Wegener's) and Sjögren's syndrome, renal diseases such as Goodpasture's syndrome, rapidly-progressing glomerulonephritis and membrano-proliferative glomerulonephritis type II, endocrine diseases such as type-I diabetes, autoimmune polyendocrinopathy-candidiasis-ectodermal dystrophy (APECED), autoimmune parathyroidism, pernicious anemia, gonad insufficiency, idiopathic Morbus Addison's, hyperthyreosis, Hashimoto's thyroiditis and primary myxedema, skin diseases such as pemphigus vulgaris, bullous pemphigoid, herpes gestationis, epidermolysis bullosa and erythema multiforme major, liver diseases such as primary biliary cirrhosis, autoimmune cholangitis, autoimmune hepatitis type-1, autoimmune hepatitis type-2, primary sclerosing cholangitis, neuronal diseases such as multiple sclerosis, myasthenia gravis, myasthenic Lambert-Eaton syndrome, acquired neuromyotony, Guillain-Barré syndrome (Müller-Fischer syndrome), stiff-man syndrome, cerebellar degeneration, ataxia, opsoklonus, sensoric neuropathy and achalasia, blood diseases such as autoimmune hemolytic anemia, idiopathic thrombocytopenic purpura (Morbus Werlhof), infectious diseases with associated autoimmune reactions such as AIDS, Malaria and Chagas disease.

The present invention can be carried out in conjunction with other treatment approaches, e.g., in combination with a second or multiple other active pharmaceutical agents, e.g., biologic or chemotherapeutic agents, or with chemoradiation. Such other active pharmaceutical agents can include, without limitation, the chemotherapeutic agents described in “Modern Pharmacology with Clinical Applications”, Sixth Edition, Craig & Stitzel, Chpt. 56, pg 639-656 (2004), herein incorporated by reference in its entirety. Such additional agent can be, but is not limited to, Interferon-α, Interferon-β, Interferon-λ, alkylating agents, antimetabolites, anti-tumor antibiotics, plant-derived products such as taxanes, enzymes, hormonal agents, miscellaneous agents such as cisplatin, monoclonal antibodies, glucocorticoids, mitotic inhibitors, topoisomerase I inhibitors, topoisomerase II inhibitors, immunomodulating agents such as interferons, cellular growth factors, cytokines, and nonsteroidal anti-inflammatory compounds (NSAID), cellular growth factors and kinase inhibitors. Other suitable classifications for chemotherapeutic agents include mitotic inhibitors, and anti-estrogenic agents.

Specific examples of suitable biological and chemotherapeutic agents include, but are not limited to, carboplatin, cisplatin, carmustine (BCNU), 5-fluorouracil (5-FU), cytarabine (Ara-C), gemcitabine, methotrexate, daunorubicin, doxorubicin, dexamethasone, irinotecan, topotecan, etoposide, paclitaxel, docetaxel, vincristine, tamoxifen, TNF-alpha, TRAIL and other members, i.e., other than TRAIL and TNF-alpha, of the TNF superfamily of molecules, interferon (in both its alpha and beta forms), thalidomide, thalidomide derivatives such as lenalidomide, melphalan, and PARP inhibitors. Other specific examples of suitable chemotherapeutic agents include nitrogen mustards such as cyclophosphamide, alkyl sulfonates, nitrosoureas, ethylenimines, triazenes, folate antagonists, purine analogs, pyrimidine analogs, anthracyclines, bleomycins, mitomycins, dactinomycins, plicamycin, vinca alkaloids, epipodophyllotoxins, taxanes, glucocorticoids, L-asparaginase, estrogens, androgens, progestins, luteinizing hormones, octreotide actetate, hydroxyurea, procarbazine, mitotane, hexamethylmelamine, carboplatin, mitoxantrone, monoclonal antibodies, levamisole, interferons, interleukins, filgrastim and sargramostim.

Useful chemotherapeutic agents include, but are not limited to, alkylating agents (e.g., cyclophosphamide, mechlorethamine, chlorambucil, melphalan), anthracyclines (e.g., daunorubicin, doxorubicin, epirubicin, idarubicin, mitoxantrone, valrubicin), cytoskeletal disruptors (e.g., paclitaxel, docetaxel), epothilones (e.g., epothilone A, epothilone B, epothilone D), inhibitors of topoisomerase I and II (e.g., irinotecan, topotecan, etoposide, teniposide, tafluposide), nucleotide analogs precursor analogs (e.g., azacitidine, azathioprine, capecitabine, cytarabine, doxifluridine, fluorouracil, gemcitabine, mercaptopurine, methotrexate, tioguanine), peptide antibiotics (e.g., bleomycin), platinum-based agents (e.g., carboplatin, cisplatin, oxaliplatin), retinoids (e.g., all-trans retinoic acid), and vinca alkaloids and derivatives (e.g., vinblastine, vincristine, vindesine, vinorelbine). In some embodiments, chemotherapeutic agents include fludarabine, doxorubicin, paclitaxel, docetaxel, camptothecin, etoposide, topotecan, irinotecan, cisplatin, carboplatin, oxaliplatin, amsacrine, mitoxantrone, 5-fluoro-uracil, or gemcitabine.

Non-steroidal anti-inflammatory drugs (NSAIDs) have been shown to induce apoptosis in colorectal cells. NSAIDs appear to induce apoptosis via the release of SMAC from the mitochondria (PNAS, Nov. 30, 2004, vol. 101:16897-16902). Therefore, the use of NSAIDs in combination with the compounds and compositions that are used in the method of the present invention may increase the activity of each drug over the activity of either drug independently.

The present invention can be carried out with co-administration of TRAIL or other chemical or biological agents which bind to and activate the TRAIL receptor(s). TRAIL has received considerable attention recently because of the finding that many cancer cell types are sensitive to TRAIL-induced apoptosis, while most normal cells appear to be resistant to this action of TRAIL. TRAIL-resistant cells may arise by a variety of different mechanisms including loss of the receptor, presence of decoy receptors, or overexpression of FLIP which competes for zymogen caspase-8 binding during DISC formation. In TRAIL resistance, a compound or composition that is used in the method of the present invention may increase tumor cell sensitivity to TRAIL leading to enhanced cell death, the clinical correlations of which are expected to be increased apoptotic activity in TRAIL resistant tumors, improved clinical response, increased response duration, and ultimately, enhanced patient survival rate. In support of this, reduction in XIAP levels by in vitro antisense treatment has been shown to cause sensitization of resistant melanoma cells and renal carcinoma cells to TRAIL (Chawla-Sarkar, et al., 2004). The Smac mimetic compounds used in the method of the present invention bind to IAPB and inhibit their interaction with caspases, therein potentiating TRAIL-induced apoptosis.

The combination of agents used in the practice of this invention can also be applied locally, such as in isolated limb perfusion. The compounds used in the method of the invention can also be applied topically, e.g., as a cream, gel, lotion, or ointment, or in a reservoir or matrix-type patch, or in an active transdermal delivery system.

The process of drug discovery typically entails screening of compounds to identify those compounds that have a desirable biological activity, e.g., binding to a certain receptor or other protein, and then, on the basis of such activity, identifying the compound as a lead for further development. Such further development can be, e.g., by chemical modification of the compound to improve its properties (sometimes referred to as lead optimization) or by putting the compound through other tests and analyses to profile the compound and thereby to further assess its potential as a drug development candidate.

In an aspect of the present invention, a compound or compounds that have been identified as Smac mimetics, e.g., based on IAP binding and degradation, such compounds are tested to determine their cIAP degradation profile and compounds that
degrade both cIAP-1 not associated with TRAF2 (free cIAP-1) and TRAF2 associated cIAP-1
degrade only TRAF2-associated cIAP-2
do not degrade cIAP-2 not bound to TRAF2 (e.g., cytoplasmic cIAP-2) as efficiently as TRAF2-bound cIAP-2,
   and are advanced to further stages of pre-clinical development, provided, of course, that they are not eliminated from consideration based on other factors, e.g., safety, potency, ease of synthesis or formulating, stability, etc.

At some point, if the process is successful, a Smac mimetic having the desired cIAP degradation profile is then selected for clinical development, i.e., human clinical trials, which are designed, ultimately, to demonstrate safety and efficacy to a level of acceptability to a drug regulatory agency. A drug regulatory agency is a governmental, or quasi-governmental, agency empowered to receive and review applications for approval to market a drug in a given jurisdiction. Examples include the U.S. Food and Drug Administration in the U.S. (“FDA”), the European Agency for the Evaluation of Medicines in the European Union (“EMEA”), and the Ministry of Health in Japan (“MOH”). In accordance with this invention, data illustrating the compound's cIAP degradation profile would be included in a dossier of information and data submitted to the regulatory agency.

If clinical development is concluded successfully, then the person or entity developing the drug will apply to a drug regulatory agency for approval to market the drug. The applicant submits information and data relating to the safety and efficacy of the compound for which approval is sought. Such data can include data indicating the mechanism by which the compound causes a particular pharmacological result. So, in relation to this invention, the applicant may submit data showing that the compound has the desired cIAP degradation profile, along with a plethora of additional information and data including, of course, the results of the clinical testing.

If the drug is approved by the regulatory agency, then the holder of the approval can begin to commercialize the drug. Part of this process includes informing patients, prescribers, insurers and healthcare providers of the characteristics of the drug. In an aspect of this invention, such marketing includes providing information about the cIAP degradation profile of the active pharmaceutical ingredient in the drug, as discussed elsewhere herein.

### EXAMPLES

### GFP-Western Blot cIAP Degradation Analysis

### Summary

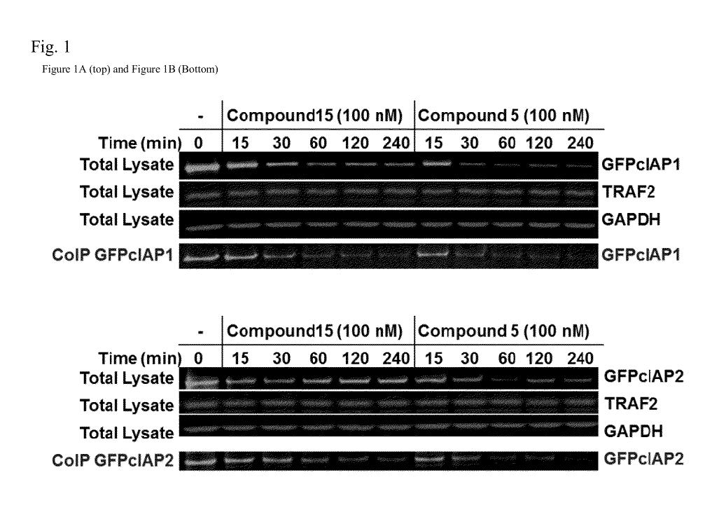

To evaluate the Smac-mimetic activity in cancer cell lines, we have developed intra-cellular green fluorescence protein (GFP)-tagged cIAP-1 and GFP-tagged cIAP-2 assays. In this assay, treatment of GFP-cIAP expressing melanoma cell line A375 with Smac mimetic, Compound 15, for two hours, results in degradation of cIAP-1 and cIAP-2 with concomitant loss of GFP-signal. (FIG. 1) The IC50's for GFP-tagged cIAP-1 and GFP-tagged cIAP-2 proteins were 22 nM and 150 nM, respectively.

It has been shown that TRAF2 strongly interacts with cIAP-1 and cIAP-2 in cells and plays an important role in transducing signals upon TNFα stimulation in cancer cells as well as normal cells. Following treatment of A375 cells with 100 nM Compound 15 for 0 to 240 minutes, cIAP-1 and cIAP-2 proteins were fractionated using anti-TRAF2 antibody by co-immunoprecipitation. The degradation kinetics of cIAP-1 and cIAP-2 (both TRAF2-associated and total) were evaluated by western blot analysis. (FIG. 1)

### Experimental Procedures

### Co-Immunoprecipitation

The stable cell lines expressing GFP-cIAP-1 or GFP-cIAP-2 were cultured in 10 cm dishes in subconfluence overnight. The cells were treated with 100 nM Compound 15 at 37° C. for the times indicated in FIG. 1, followed by lysis with a lysis buffer containing 20 mM Tris-HCl (pH7.5), 150 mM NaCl, 0.5% Triton X-100, 10% Glycerol, 2 mM EDTA, 50 mM NaF, 25 mM β-Glycerophosphate, 0.2 mM NaVO₄, mM Na-pyrophosphate and protease cocktail (Roche). The lysate was cleared by centrifugation (14000 rpm) at 4° C. for 10 minutes and the supernatant was subjected to the co-immunoprecipitation experiment. The protein concentration of the supernatant was measured by using Bradford reagent (Sigma). Co-immunoprecipitation was carried out with 600 μg of the total protein and 1 μg of anti-TRAF 2 antibody (Cell Signaling). After the incubation at 4° C. for overnight, 25 μL proteinA/G beads (Invitrogen) was added to precipitate the immunocomplex. The beads were washed with the lysis buffer three times, followed by dissolving with 25 μL SDS-Loading buffer (Invitrogen). Proteins were fractionated by 8-16% Tris-Glycine gradient gel and transferred to Immobilon-FL membrane for Western blot analysis.

### Western Blot Analysis

The membrane was soaked with the blocking buffer (LICOR) for 30 minutes, followed by incubation with the primary antibodies, cIAP-1 and cIAP-2 (R&D Systems), at 4° C. for overnight. After the incubation, the membrane was washed three times with TBS-T buffer (Invitrogen) and incubated with IRDye anti-goat antibody (LICOR) at 37° C. for 45 minutes. Following 5 times washings, the membrane was scanned by The Odyssey Infrared Imaging System (LICOR).

### Results

FIG. 1 shows the results of the western blot analysis:

1) Rapid and complete degradation of both cIAP-1 not bound to TRAF2 (e.g., cytoplasmic cIAP-1) and TRAF2-bound cIAP-1 (FIG. 1a);
2) Degradation kinetics of TRAF2-bound cIAP-1 and total cIAP-1 were identical (FIG. 1a);
3) Complete degradation of TRAF2-bound cIAP-2 occurred while minimal degradation of the total cIAP-2 (FIG. 1b).

These data show that the activity of Compound 15 is comparable when cIAP-1 or cIAP-2 are associated with TRAF2. However, Compound 15 very potently triggers degradation of cIAP-1 [whether bound to TRAF2 or not] while only TRAF2-associated cIAP-2 is degraded in the presence of Compound 15.

Similar analysis was conducted using a pan-IAP antagonist, namely, an analog of Compound 15 wherein R5 is —R—CH(OCH3)CH3, hereinafter referred to as Compound 5. This compound degraded GFP-tagged cIAP-1 and GFP-tagged cIAP-2 with comparable activity (IC50). When the IAPB were fractionated by co-immunoprecipitating TRAF2, both cIAP-1 and cIAP-2 degradation kinetics were similar (FIGS. 1A and 1B).

These results are apparent in FIG. 1. Note that in FIG. 1A, the top row, i.e., row 1, is cytoplasmic cIAP-1, i.e., free cIAP-1 and the bottom row, i.e., row 4, is cIAP-1 bound to TRAF2. Row 2 (TRAF2) and row 3 (GAPDH) are controls. Note that the extent of degradation of cytoplasmic cIAP-1 is visually approximately the same as the extent of degradation of cIAP-1 bound to TRAF2 in the case of Compound 15 and also in the case of Compound 5.

In contrast, in FIG. 1B, cytoplasmic cIAP-2 (row 1) is substantially non-degraded and TRAF2-bound cIAP-2 (row 4) is degraded in the case of Compound 15 whereas both cytoplasmic cIAP-2 and cIAP-2 bound to TRAF2 are degraded in the case of Compound 5.

As reported in US2011003877, which is incorporated herein in its entirety by reference as though fully set forth, Compound 5 is less well tolerated in rats compared to Compound 15 while possessing comparable antitumor activity. Despite their differences in cIAP-2 degradation, both compounds showed comparable xenograft activity suggesting that degradation of free cIAP-2 is not necessary for tumor regression, but presence of cytoplasmic cIAP-2 contributes to better therapeutic index. TRAF2-bound cIAP 1 and cIAP2 were resistant to monovalent Smac-Mimetics

In the following experiments we sought to: 1) compare the activities of birinapant versus monovalent Smac-Mimetic compounds and 2) determine if there was evidence to support differential activity of these compounds on total versus TRAF2-bound cIAPs in cells.

Since TNF Receptor-Associated Factor 2 (TRAF2) is known to interact with cIAP 1 and cIAP2, and plays a role in recruitment of cIAP 1 and cIAP2 to TNFR complex upon TNFα stimulation, we performed immunoprecipitation experiments using anti-TRAF2 antibody to evaluate the activity of Smac-Mimetics against TRAF2-bound cIAPs. This was compared with ‘total’ cIAPs within cells. To compare the different classes of Smac-Mimetic, birinapant was compared with GT14433 as a representative example of monovalent Smac-Mimetics. GT14433 has the following structure:

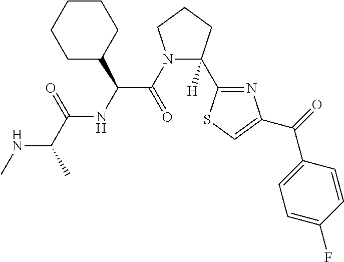 [SMILES: CN[C@@H](C)C(=O)N[C@H](C(=O)N1CCC[C@H]1c1nc(C(=O)c2ccc(F)cc2)cs1)C1CCCCC1]

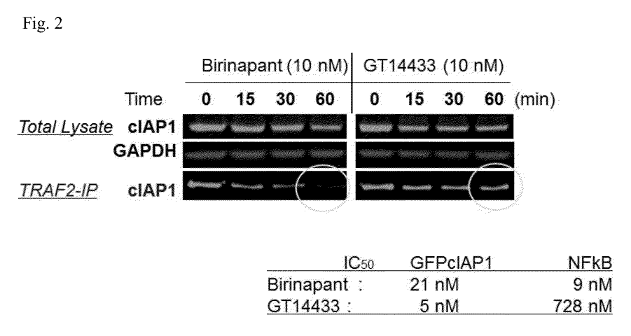

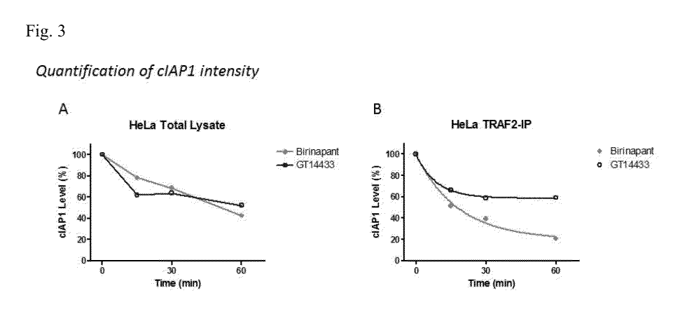

In the first immunoprecipitation experiment, we used a low concentration (10 nM) of birinapant to treat HeLa cells. As shown in FIG. 2, total cIAP1 level in HeLa cells treated with birinapant decreased slowly and modestly, and in a time-dependent manner. In fact, the majority of total cIAP1 (−50%) was still intact in birinapant-treated cells (FIG. 3, panel A). However, there was a difference in degradation of TRAF2-bound cIAP1 when cells were treated with birinapant: approximately 80% of TRAF2-bound cIAP1 was degraded. Experiments performed in Jurkat cells also showed similar results.

In contrast, when cells were treated with GT14433, there was no evidence of TRAF2-selective degradation of cIAP1. HeLa and Jurkat cells were treated with 10 nM GT14433. This resulted in a modest decrease in cIAP1 of ˜50% (FIG. 2, 3A), with no evidence of a selective loss of TRAF2-bound cIAP1: the TRAF2-bound cIAP1 was also degraded approximately 50% (FIG. 3B).

In order to rule out the possibility of these results being a reflection simply of the concentration of compounds used in the experiments described, we performed a similar experiment using 100 nM of either birinapant or GT14433. This concentration is well above the IC50 for degradation of total cIAPs measured by the GFP assay. Birinapant induced the degradation of both total cIAP 1 and TRAF2-bound cIAP 1. In contrast, GT14433 and other monovalent Smac-Mimetics did not cause complete degradation of either total or TRAF2 bound cIAP 1.

The above data supports the discovery that a Smac mimetic that has the following properties:
degrades both cIAP-1 not associated with TRAF2 (e.g., cytoplasmic cIAP-1) and TRAF2-associated cIAP-1;
degrades TRAF2-associated cIAP-2;
does not degrade cIAP-2 not associated with TRAF2 (e.g., cytoplasmic cIAP-2) as efficiently as TRAF2-bound cIAP-2 and does not substantially degrade cIAP-2 not associated with TRAF2 (e.g., cytoplasmic cIAP-2),
   is better tolerated at doses of comparable efficacy in comparison to a Smac mimetic that:
degrades both cIAP-1 not associated with TRAF2 (e.g., cytoplasmic cIAP-1) and TRAF2-associated cIAP-1
degrades both cIAP-2 not associated with TRAF2 (e.g., cytoplasmic cIAP-2) and TRAF2-associated cIAP-2 to a comparable extent.

Such Smac mimetic is particularly well tolerated and well suited for use in a pharmaceutical composition, as well as in a method for treating a proliferative disorder or an autoimmune disorder. In particular, the pharmaceutical composition of the invention for the treatment of a proliferative disorder, which comprises an effective amount of such Smac mimetic in addition to at least one pharmaceutically acceptable excipient, can improve therapeutic index by reducing toxicities. The reduced toxicities include, e.g., one of, or any combination of one or more of: reduced body weight loss, decreased incidence of mortality, reduced bone marrow hypocellularity of the erythroid series, reduced hypercellularity of the myeloid series, reduced hypertrophy and hyperplasia of megakaryocytes, reduced diffuse hypertrophy/hyperplasia of Type 2 pneumocytes decreased lethargy, more regular respiration, and lessened increase in heart rate.

It is understood that the examples and embodiments described herein are for illustrative purposes only and that various modifications or changes in light thereof will be suggested to persons skilled in the art and are to be included within the spirit and purview of this application and the scope of the appended claims.

1. A method of treating a patient suffering from a proliferative disorder that comprises:
a) assaying a Smac mimetic in a cIAP degradation assay to determine whether or not the Smac mimetic induces degradation of cIAP-1 not bound to TRAF2, of cIAP-1 bound to TRAF2, of cIAP-2 not bound to TRAF2 and of cIAP-2 bound to TRAF2
and, if the Smac mimetic degrades cIAP-1 not bound to TRAF2, cIAP-1 bound to TRAF2 and cIAP-2 bound to TRAF2 and either does not degrade cIAP-2 not bound to TRAF2 or degrades cIAP-2 not bound to TRAF2 to a lesser extent than cIAP-2 bound to TRAF2, then
b) internally administering an effective amount of a Smac mimetic having the same chemical structure as the assayed Smac mimetic, or a pharmaceutically acceptable salt thereof, to the patient.

2. A method of preparing a pharmaceutical composition comprising a Smac mimetic for treating a patient suffering a proliferative disorder that comprises:
a) assaying a Smac mimetic in a cIAP degradation assay to determine whether or not the Smac mimetic induces degradation of cIAP-1 not bound to TRAF2, of cIAP-1 bound to TRAF2, of cIAP-2 not bound to TRAF2 and of cIAP-2 bound to TRAF2 and, if the Smac mimetic degrades cIAP-1 not bound to TRAF2, cIAP-1 bound to TRAF2 and cIAP-2 bound to TRAF2 and either does not degrade cIAP-2 not bound to TRAF2 or degrades cIAP-2 not bound to TRAF2 to a lesser extent than cIAP-2 bound to TRAF2, then
b) formulating a Smac mimetic having the same chemical structure as the assayed Smac mimetic, or a pharmaceutically acceptable salt thereof, into a pharmaceutical composition.

3. A method of screening for a Smac mimetic for use in the treatment of a proliferative disorder in a patient, said method comprising:
assaying a Smac mimetic in a cIAP degradation assay to determine whether or not the Smac mimetic induces degradation of cIAP-1 not bound to TRAF2, of cIAP-1 bound to TRAF2, of cIAP-2 not bound to TRAF2 and of cIAP-2 bound to TRAF2.

4. The method of claim 3 in which the Smac mimetic is bivalent.

5. The method of claim 1 wherein the proliferative disorder is a cancer, a benign proliferative disorder, or an autoimmune disorder.

6. The method of claim 5 wherein the proliferative disorder is a cancer selected from the group consisting of: lung adenocarcinoma, pancreatic cancer, colon cancer, ovarian cancer, breast cancer, mesothelioma, peripheral neuroma, bladder cancer, glioblastoma, melanoma, adrenocortical carcinoma, AIDS-related lymphoma, anal cancer, bladder cancer, meningioma, glioma, astrocytoma, breast cancer, cervical cancer, chronic myeloproliferative disorders (e.g., chronic lymphocytic leukemia, chronic myelogenous leukemia), colon cancer, endocrine cancers, endometrial cancer, ependymoma, esophageal cancer, Ewing's sarcoma, extracranial germ cell tumors, extragonadal germ cell tumors, extrahepatic bile duct cancer, gallbladder cancer, gastric cancer, gastrointestinal carcinoid tumors, gestational trophoblastic tumors, hairy cell leukemia, Hodgkin lymphoma, non-Hodgkin lymphoma, hypopharyngeal cancer, intraocular melanoma, islet cell carcinoma, Kaposi sarcoma, laryngeal cancer, leukemia, acute lymphoblastic leukemia, acute myeloid leukemia, lip cancer, oral cavity cancer, liver cancer, male breast cancer, malignant mesothelioma, medulloblastoma, melanoma, Merkel cell carcinoma, metastatic squamous neck cancer, multiple myeloma and other plasma cell neoplasms, mycosis fungoides and the Sezary syndrome, myelodysplastic syndromes, nasopharyngeal cancer, neuroblastoma, non-small cell lung cancer, small cell lung cancer, oropharyngeal cancer, bone cancers, including osteosarcoma and malignant fibrous histiocytoma of bone, ovarian epithelial cancer, ovarian germ cell tumors, ovarian low malignant potential tumors, pancreatic cancer, paranasal sinus cancer, parathyroid cancer, penile cancer, pheochromocytoma, pituitary tumors, prostate cancer, rectal cancer, renal cell cancer, retinoblastoma, rhabdomyosarcoma, salivary gland cancer, skin cancer, small intestine cancer, soft tissue sarcoma, supratentorial primitive neuroectodermal tumors, pineoblastoma, testicular cancer, thymoma, thymic carcinoma, thyroid cancer, transitional cell cancer of the renal pelvis and ureter, urethral cancer, uterine sarcoma, vaginal cancer, vulvar cancer, and Wilm's tumor and other childhood kidney tumors.

7. The method of claim 5 wherein the proliferative disorder is a cancer selected from the group consisting of: sarcomas, bladder cancer, ovarian cancer, breast cancer, brain cancer, pancreatic cancer, colon cancer, blood cancer, skin cancer, lung cancer, and bone cancer.

8. The method of claim 5 wherein the proliferative disorder is a cancer selected from colorectal cancer, renal carcinoma, ovarian carcinoma, pancreatic carcinoma, prostate carcinoma, breast carcinoma, melanoma, gliobastoma, acute myeloid leukemia, small cell lung cell carcinoma, non-small cell lung carcinoma, rhabdomyosarcoma, and basal cell carcinoma.

9. The method of claim 5 wherein the proliferative disorder is an autoimmune disease that is caused or exacerbated by abnormal regulation of apoptosis, selected from the group consisting of: systemic lupus erythematosus, psoriasis, and idiopathic thrombocytopenic purpura (Morbus Werlhof).

10. A compound that is a Smac mimetic that in a cIAP degradation assay:
induces degradation of cIAP-1 not bound to TRAF2, cIAP-1 bound to TRAF2, and cIAP-2 bound to TRAF2 and either does not degrade cIAP-2 not bound to TRAF2 or degrades cIAP-2 not bound to TRAF2 to a lesser extent than cIAP-2 bound to TRAF2, excluding Compound 15.

11. The compound of claim 10 that is a bivalent Smac mimetic.

12. The compound of claim 10 that has the formula
[P1-P2-P3-P4]-L-[P1′-P2′-P3′-P4′]
wherein P1-P2-P3- and P1′-P2′-P3′-correspond to the N-terminal Ala-Val-Pro- of mature Smac and P4 and P4′ correspond to Phe, Tyr, Ile, or Val and L is a linking group, or bond, covalently linking [P1-P2-P3-P4] to [P1′-P2′-P3′-P4′].

13. The compound of claim 10 that has the formula
[P1-P2-P3-P4]-L-[P1′-P2′-P3′-P4′]
wherein
P1 and P1′ are NHR1-CHR2—C(O)—;
P2 and P2′ are —NH—CHR3—C(O)—;
P3 and P3′ are pyrrolidine, pyrrolidine fused to a cycloalkyl, or pyrrolidine fused to a heterocycloalkyl having a —N— heteroatom, and wherein the pyrrolidine of P3/P3′ is bound to P2/P2′ by an amide bond;
P4 and P4′ are -M-Qₚ-R⁷;
R¹ is —H or —CH3;
R² is —CH3 or —CH2CH3;
R³ is C2-6 alkyl, C2-6 alkoxy, cycloalkyl, heterocycloalkyl, aryl or heteroaryl, optionally substituted in each case;
M is a covalent bond, C1-6 alkylene, substituted C1-C6 alkylene such as but not limited to —C(O)—;
Q is a covalent bond, C1-6 alkylene, substituted C1-C6 alkylene, —O— or —NR⁸—, provided that M is not a covalent bond if: (1) -M- is bound directly to the 2 position of a P3/P3′ pyrrolidine or to a heteroatom in a P3/P3′ pyrrolidine-heterocycloalkyl bicycle and (2) Q is —O— or —NR⁸—;
p is 0 or 1;
R⁷ is cycloalkyl, cycloalkylaryl, aryl or heteroaryl, optionally substituted in each case;
R⁸ is —H or C1-6 alkyl;
L is a linking group, or bond, covalently linking [P1-P2-P3-P4] to [P1′-P2′-P3′-P4′].
